(19) United States
(12) Patent Application Publication
(10) Pub. No.: US 2005/0261773 A1
(43) Pub. Date: Nov. 24, 2005

(54) Lateral-approach artificial disc replacements

(76) Inventor: Bret A. Ferree, Cincinnati, OH (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 10/438,605

(22) Filed: May 15, 2003

Related U.S. Application Data
(63) Continuation-in-part of application No. 10/413,028, filed on Apr. 14, 2003.
(60) Provisional application No. 60/378,132, filed on May 15, 2002.

(51) Int. Cl. A61F 2/44

(52) U.S. Cl. 623/17.16; 623/17.11

(57) ABSTRACT

Artificial disc replacements (ADRS) are configured for implantation using a lateral, anterior-lateral, or posterior-lateral approach. A first component having a first segment resides in the disc space for articulation purposes, with a second segment adapted for fixation to the lateral outer surface of one of the vertebral bodies. A second component having a first segment resides in the disc space for articulation purposes, with a second segment adapted for fixation to the lateral outer surface of the other vertebral body. The first segments of the two components may articulate against one another without a spacer, or a spacer forming a mobile bearing may be disposed between the first segments of the two components. In the preferred embodiment, one or both of the two components are in the form of bent plates such that the second segment is positioned against a lateral wall for fixation. In the preferred embodiment, screws are used through the second segment and into a vertebral body. The screws are located in different vertical locations, and may diverge or converge vertically or horizontally to resist pull-out.

### REFERENCE TO RELATED APPLICATIONS

This application claims priority from U.S. Provisional Patent Application Ser. No. 60/378,132, filed May 15, 2002; and is a continuation-in-part of U.S. patent application Ser. No. 10/413,028, filed Apr. 14, 2003. The entire contents of both applications are incorporated herein by reference.

### FIELD OF THE INVENTION

This invention relates generally to spine surgery and, more particularly, to artificial disc replacements based upon a lateral approach.

### BACKGROUND OF THE INVENTION

Premature or accelerated intervertebral disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The nucleus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, replace either the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

Generally “total disc replacements” (TDRs) are performed through the abdomen in an anterior approach to the spine. “Nucleus replacements” (NR), in contrast, are generally performed through a posterior approach. A few surgeons have tried a lateral approach to insert NR devices under the belief that NRs placed from a lateral approach may be less likely to extrude. However, a NR extrusion rate of up to 50 percent has been reported with the posterior approach.

### SUMMARY OF THE INVENTION

This invention improves upon existing techniques by facilitating artificial disc replacements (ADR) through a lateral, anterior-lateral, or posterior-lateral approach. Broadly, ADRs according to the invention include a first component having a first segment that resides in the disc space for articulation purposes and a second segment adapted for fixation to the lateral outer surface of one of the vertebral bodies, and a second component having a first segment that resides in the disc space for articulation purposes and a second segment adapted for fixation to the lateral outer surface of the other vertebral body.

The first segments of the two components may articulate against one another without a spacer, or a spacer forming a mobile bearing may be disposed between the first segments of the two components. The spacer is preferably polyethylene, though other rigid and compressible/resilient spacers may be used, including other polymers and encased foams and gels, including hydrogels. The spacer is preferably wider laterally than anterior to posterior, and one or both of the two components include physical features to retain the spacer within the disc space.

In the preferred embodiment, one or both of the two components are in the form of bent plates such that the second segment is positioned against a lateral wall for fixation. In the preferred embodiment, screws are used through the second segment and into a vertebral body. The screws are located in different vertical locations, and may diverge or converge vertically or horizontally to resist pull-out. The screws may be locked to the second segment(s), or may project through a vertebral body. One or both of the components, and the spacer if used, may be rounded or otherwise shaped to fit through a working cannula.

In some embodiments, the region of articulation, with or without a spacer, may be positioned more posterior than anterior. For example, the region of articulation may vary from posterior to anterior depending upon the vertebral level. In particular, the region of articulation may be positioned more posteriorly in the disc space at the L5/S1 level than the L4/L5 level. Additionally, the region of articulation may be at least partially non-congruent to permit a certain degree of translation.

The invention offers several important advantages. For one, the thick anterior longitudinal ligament (ALL) is preserved. Anterior approaches to the disc sacrifice the ALL. Since the ALL limits spinal extension and extension forces, it may help prevent extension forces on ADRs. The ALL may help prevent ADR extrusion. Disc space distraction tightens the ALL. Thus, once inserted, counter tension by the ALL may help to hold an ADR securely in place. Secure placement facilitates bone ingrowth.

The great vessels must be manipulated during an anterior approach. Furthermore, bulky devices cannot be left against the great vessels. Death from aneurysms caused by erosion of the great vessels against spinal devices has been reported. Lateral ADR insertion allows the use of larger plate-like extension on the device, as the great vessels lie over the anterior portion of the spine, not the lateral portion. The larger plate-like extensions, with more screws, hold the ADR more securely in place.

Furthermore, minimally invasive techniques have been developed for lateral approaches to the spine, enabling patients to recover more quickly.

### BRIEF DESCRIPTION OF THE DRAWINGS

FIG. 1 is an A-P view of an artificial disc replacement using a lateral approach according to the invention;

FIG. 1B is a lateral view of the device of FIG. 1A showing how the anterior longitudinal ligament (ALL) remains in tact using approaches according to this invention.

FIG. 1C is a lateral view of an alternative configuration illustrating a constrained configuration which allows for flexion and extension.

FIG. 2A illustrates an ADR in flexion;

FIG. 2B illustrates an ADR in extension.

FIG. 3A begins a series of drawings which shows the way in which an ADR according to this invention is installed from a lateral approach;

FIG. 3B shows both the insertion of the spacer and bottom plate;

FIG. 4 shows a more posterior positioning to facilitate greater flexion, for example.

FIG. 5 is a coronal cross section of a different embodiment of the invention, wherein a mobile spacer is shaped to allow for a certain degree of lateral bending;

FIG. 6 is a lateral view of the embodiment of the ADR drawn in FIG. 5;

FIG. 7A is a coronal cross-section of an alternative embodiment of the invention, wherein a spacer component articulates through line contact with the ADR endplates;

FIG. 7B is a sagittal cross-section of the embodiment of the ADR shown in FIG. 7A;

FIG. 7C is a lateral view of the ADR shown in FIG. 7A, showing how the ADR EPs may contain screw holes.

FIG. 8A is an anterior view of an alternative embodiment of a lateral-approach ADR including two components without a separate spacer or mobile bearing;

FIG. 8B is a lateral view of the embodiment of the ADR drawn in FIG. 8A; and

FIG. 9 shows how an entire ADR may be installed laterally with proper distraction.

### DETAILED DESCRIPTION OF THE INVENTION

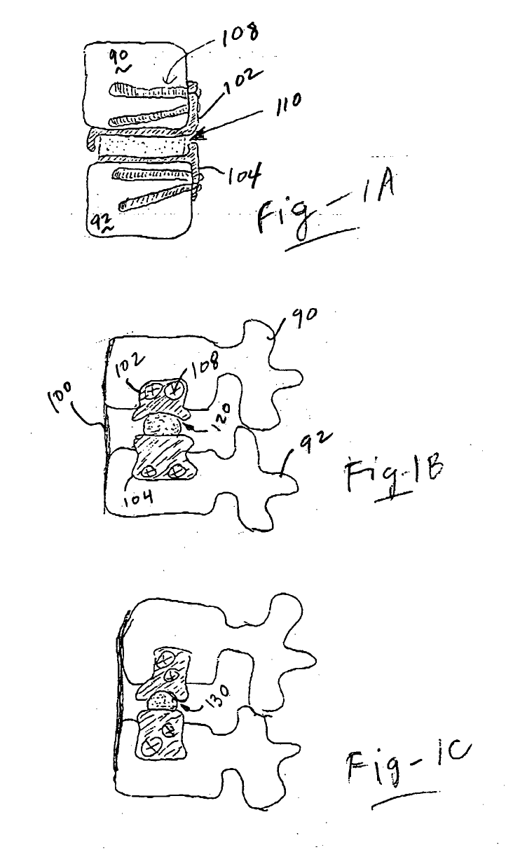

FIG. 1A is an A-P view of an artificial disc replacement using a lateral approach according to the invention. In the preferred embodiments, ADRs according to the invention feature relatively large, plate-like extensions fastened to the vertebrae 90, 92. These large plate-like extensions 102, 104 allow screws such as 108 to diverge or converge for greater pull-out strength. A spacer is used between these extensions. A polyethylene cylinder 110 is preferably used to avoid the thin sections of polyethylene found in some ADR designs. Thin sections of polyethylene risk fracture and the need for replacement as the poly becomes thin from wear. The ADR can be inserted in parts through a working cannula during minimally invasive surgery.

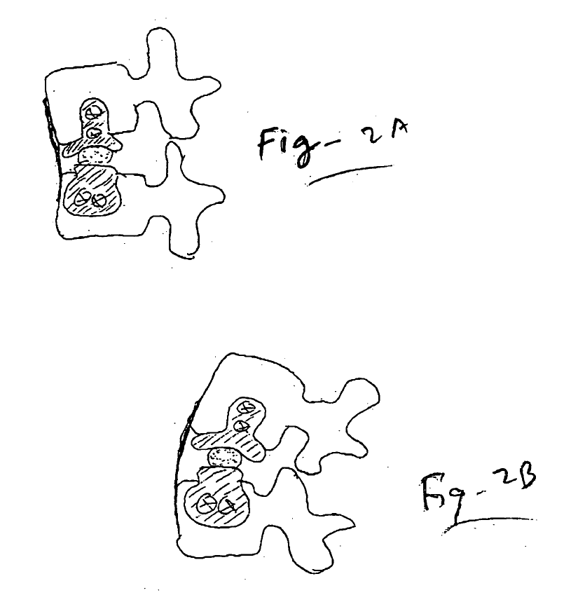

FIG. 1B is a lateral view of the device of FIG. 1A showing how the anterior longitudinal ligament (ALL, 100) remains in tact using approaches according to this invention. The system is semi-constrained, allowing flexion extension and limited translation. FIG. 1C is a lateral view of an alternative configuration illustrating a constrained configuration which allows for flexion and extension. In these embodiments, the vertebrae and end plates, preferably metal, rotate around a central cylinder during flexion and extension. FIG. 2A illustrates the ADR in flexion, and FIG. 2B illustrates the ADR in extension.

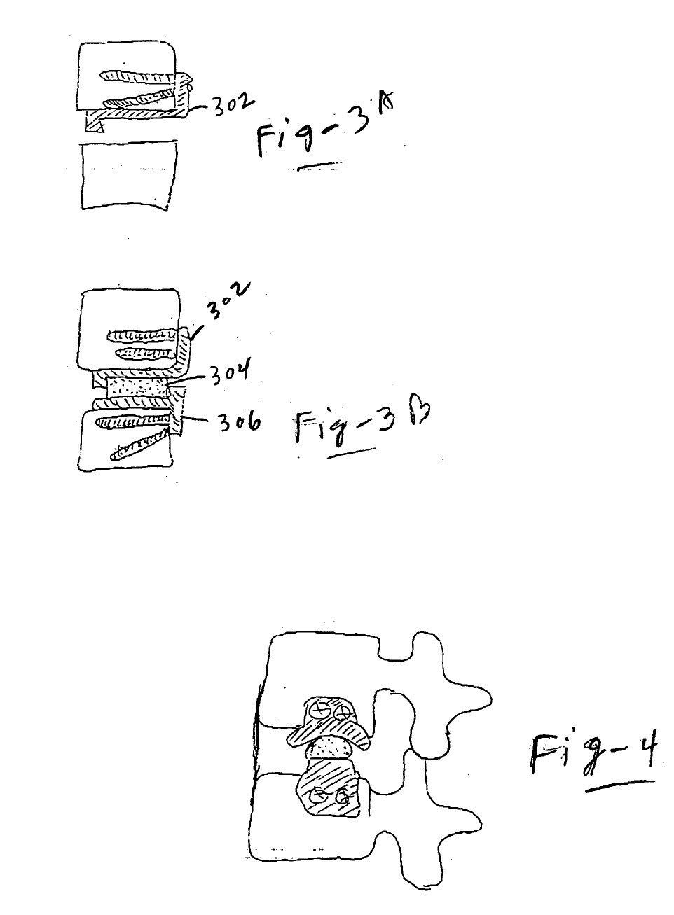

FIG. 3A begins a series of drawings which shows the way in which an ADR according to this invention is installed from a lateral approach. FIG. 3A illustrates the initial insertion of a top plate 302. FIG. 3B shows both the insertion of the spacer 304 and bottom plate 306. The device may be placed more anteriorly or posteriorly depending upon the desired degree of flexion. FIG. 4 shows a more posterior positioning to facilitate greater flexion, for example.

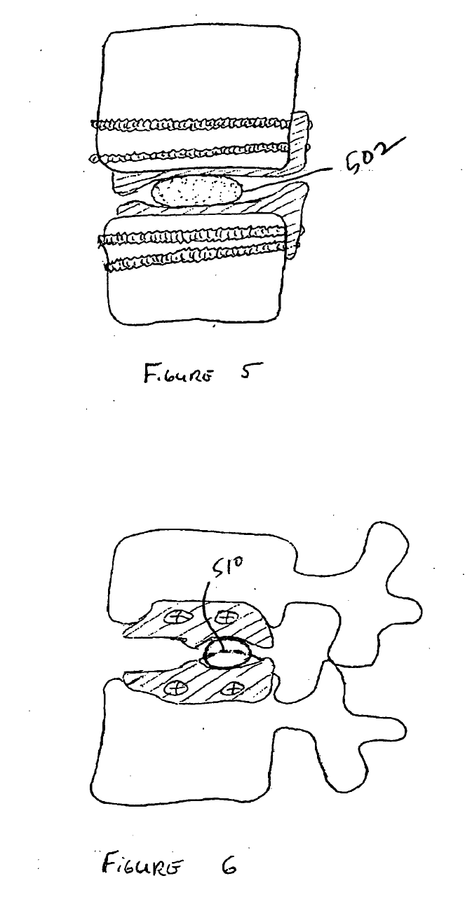

FIG. 5 is a coronal cross section of a different embodiment of the invention, wherein the mobile spacer 502 is shaped to allow for a certain degree of lateral bending. FIG. 6 is a lateral view of the embodiment of the ADR drawn in FIG. 5. Note that the surface area of the ADR EP has been increased. The ADR EPs can be designed to place the mobile bearing at different locations from anterior to posterior in the disc space. The mobile bearing retention component on the lower ADR EP is outlined by the dotted line 510 to better illustrate the articulating surface of the lower ADR EP.

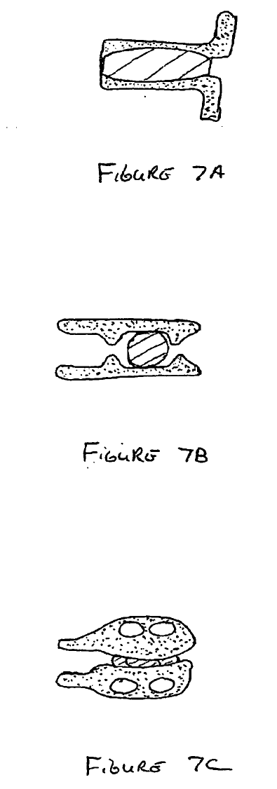

FIG. 7A is a coronal cross-section of an alternative embodiment of the invention, wherein the spacer component articulates through line contact with the ADR endplates. FIG. 7B is a sagittal cross-section of the embodiment of the ADR shown in FIG. 7A. The spacer component translates within a range allowed by the ADR EPs. The ADR EPs articulate with the spacer component. The ADR EPs also translate over the spacer component. FIG. 7C is a lateral view of the ADR shown in FIG. 7A, showing how the ADR EPs may contain screw holes.

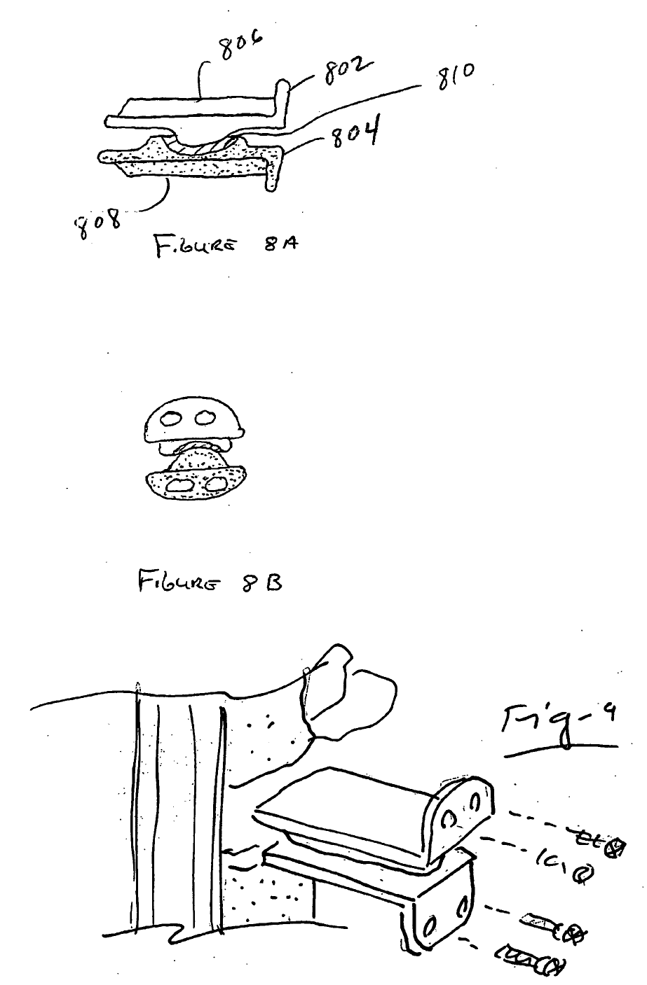

FIG. 8A is an anterior view of an alternative embodiment of a lateral-approach ADR including two components 802, 804 without a separate spacer or mobile bearing. FIG. 8B is a lateral view of the embodiment of the ADR drawn in FIG. 8A. Both components can be screwed to the lateral aspect of the spine. Optionally, as with all other embodiments disclosed herein, laterally directed keels 806, 808 may be used on the top and bottom of the ADR. The two components articulate to allow spinal motion. The two components may be metal, ceramic, or combinations thereof, and may optionally include a bonded or treated surface 810 to improve wear. The shape of the articulating surfaces may be simple or more complex, depending upon the level of the spine, degree of flexion, lateral bending, and so forth. For example, the saddle-shaped joint described in my co-pending U.S. patent application Ser. No. 10/413,028 may be used. Depending upon the design, ADRs may be installed according to the invention in a sequence of upper component followed by lower (or vice versa) for a spacerless design, or upper, lower, and spacer, in any order appropiate to design, vertebral level, or other factors. FIG. 9 shows how an entire ADR may be installed laterally with proper distraction.

1. An artificial disc replacement (ADR) configured for lateral installation with respect to adjacent vertebral bodies, each having anterior and posterior portions, a lateral outer surface and an endplate facing a disc space, comprising:
a first component having a first segment that resides in the disc space for articulation purposes and a second segment adapted for fixation to the lateral outer surface of one of the vertebral bodies; and
a second component having a first segment that resides in the disc space for articulation purposes and a second segment adapted for fixation to the lateral outer surface of the other vertebral body.

2. The ADR of claim 1, wherein the first segments of the two components articulate against one another without a spacer.

3. The ADR of claim 1, further including a spacer disposed between the first segments of the two components.

4. The ADR of claim 3, wherein the spacer is polyethylene.

5. The ADR of claim ˜3, wherein the spacer forms a mobile bearing.

6. The ADR of claim 3, wherein the spacer is wider laterally than anterior to posterior.

7. The ADR of claim 3, wherein one or both of the two components include physical features to retain the spacer within the disc space.

8. The ADR of claim 3, wherein one or both of the two components are in the form of bent plates such that the second segment is positioned against a lateral wall for fixation.

9. The ADR of claim 8, wherein screws are used through the second segment and into a vertebral body.

10. The ADR of claim 9, wherein the screws are located in different vertical locations.

11. The ADR of claim 9, wherein the screws diverge or converge vertically or horizontally to resist pull-out.

12. The ADR of claim 9, wherein the screws are locked to the second segment.

13. The ADR of claim 9, including screws that project through a vertebral body.

14. The ADR of claim 9, wherein one or both of the components are rounded or otherwise shaped to fit through a working cannula.

15. The ADR of claim 1, including a region of articulation that is more posterior than anterior.

16. The ADR of claim 1, including a region of articulation that varies from posterior to anterior depending upon the vertebral level.

17. The ADR of claim 16, wherein the region of articulation is positioned more posteriorly in the disc space at the L5/S1 level than the L4/L5 level.

18. The ADR of claim 3, wherein the spacer is positioned more posterior than anterior.

19. The ADR of claim 3, wherein the position of the spacer varies from posterior to anterior depending upon the vertebral level.

20. The ADR of claim 18, wherein the spacer is positioned more posteriorly in the disc space at the L5/S1 level than the L4/L5 level.

21. The ADR of claim 1, wherein the region of articulation is at least partially non-congruent to permit a certain degree of translation.

22. A method of installing an artificial disc replacement (ADR) into the disc space between adjacent vertebral bodies, each having anterior and posterior portions, a lateral outer surface and an endplate facing a disc space, the method comprising the steps of:
installing first and second components, each having a first segment that resides in the disc space for articulation purposes and a second segment positioned adjacent the lateral outer surface of one of the vertebral bodies; and
fastening the second segments to the lateral portions of the respective vertebral bodies.

23. The method of claim 22, wherein the second segments are fastened to the lateral portions of the respective vertebral bodies using screws that penetrate the respective vertebral bodies.

24. The method of claim 22, wherein the screws converge or diverge to prevent back-out.

25. The method of claim 22, wherein the components are installed simultaneously.

26. The method of claim 22, including a third component that functions as a spacer between the other two components.
